(19) United States
(12) Patent Application Publication
(10) Pub. No.: US 2026/0083419 A1
(43) Pub. Date: Mar. 26, 2026

(54) CHARACTERIZATION OF A PERFUSION DEFECT

(71) Applicant: Siemens Healthineers AG, Forchheim (DE)

(72) Inventors: Michael SUEHLING, Erlangen (DE); Markus JUERGENS, Adelsdorf (DE); Michael GRASRUCK, Eckental Forth (DE)

(73) Assignee: Siemens Healthineers AG, Forchheim (DE)

(21) Appl. No.: 19/335,177

(22) Filed: Sep. 22, 2025

(30) Foreign Application Priority Data
Sep. 23, 2024 (EP) 24201811.7

(51) Int. Cl. A61B 6/50; A61B 6/00; A61B 6/03; G06T 7/00; G06T 7/11; G06T 7/62

(52) U.S. Cl. CPC A61B 6/507; A61B 6/035; A61B 6/5217; G06T 7/0012; G06T 7/11; G06T 7/62; G06T 2207/10081; G06T 2207/20081; G06T 2207/30061; G06T 2207/30104

(57) ABSTRACT

For imaging-based characterization of a perfusion defect in a vessel structure for blood supply of an organ of a patient, medical imaging data is received, wherein the medical imaging data includes energy resolved CT imaging data. A blood stream obstructing object in the vessel structure is detected based on the medical imaging data. A perfusion defect score for a target region of the at least one organ, whose blood perfusion is potentially affected by the obstructing object, is determined depending on the energy resolved CT imaging data.

### CROSS-REFERENCE TO RELATED APPLICATION(S)

The present application claims priority under 35 U.S.C. § 119 to European Patent Application No. 24201811.7, filed Sep. 23, 2024, the entire contents of which are incorporated herein by reference.

### FIELD

One or more example embodiments of the present invention are directed to a computer-implemented method for imaging-based characterization of a perfusion defect in a vessel structure for blood supply of at least one organ of a patient, a corresponding data processing system and a corresponding non-transitory computer program product.

One or more example embodiments of the present invention are in the field of medical imaging analysis, which is used for of creating data based representations, in particular visual representations, of the interior of a body based on the detection of physical signals and imaging data-based analysis of the resultant image data to support analysis, in particular visual representation, of the function of organs or tissues.

### BACKGROUND

A pulmonary embolism is a potentially life-threatening condition that may occur when a blood clot, also denoted as thrombus, forms in one of the pulmonary arteries. The clot can block or partially obstruct the blood flow to the lungs, leading to various symptoms and complications. In medical imaging data, for example computed tomography, CT, imaging data, a pulmonary embolism may for example appear as a filling defect or an area of decreased contrast enhancement within the pulmonary arteries, for example.

Algorithms based on machine learning models, MLMs, to automatically detect pulmonary artery blood clots from medical imaging data are known. For example, the publication P. A. Grenier et al.: “Deep Learning-Based Algorithm for Automatic Detection of Pulmonary Embolism in Chest CT Angiograms”, Diagnostics (Basel), 2023, 13(7) uses artificial neural networks, ANNs, to detect such clots in CT angiography, CTA, imaging data.

However, merely detecting the presence and position of the blood clot does not necessarily allow to reliably estimate the criticality of resulting perfusion defects.

The publication E. A. Boyden: “Segmental Anatomy of the Lungs. A Study of the Patterns of the Segmental Bronchi and Related Pulmonary Vessels”, The Blakiston Division, McGraw-Hill Book Company, Inc., New York, 1955, describes a hierarchical classification of bronchi.

The publication H. Koike et. al.: “Quantification of lung perfusion blood volume (lung PBV) by dual-energy CT in patients with chronic thromboembolic pulmonary hypertension (CTEPH) before and after balloon pulmonary angioplasty (BPA): Preliminary results.”, Eur. J. Radiol., 2016, 85(9),1607, describes how the lung perfusion blood volume, PBV, may be quantified by computing the ratio of the lung PBV to the pulmonary artery enhancement in dual energy CT, DECT, imaging data.

In the publication by O. Ronneberger et al.: “U-Net: Convolutional Networks for Biomedical Image Segmentation” (arXiv:1505.04597), the U-Net architecture is described, a widely used CNN architecture for image segmentation, which may, however, also be used for other computer vision tasks, in particular image-to-image tasks.

### SUMMARY

It is an objective of the one or more example embodiments of the present invention to provide a possibility to automatically characterize a perfusion defect in our vessel structure for blood supply of at least one organ of a patient.

At least this objective is achieved by the subject matter of the independent claim(s). Further implementations and preferred embodiments are subject matter of the dependent claims.

one or more example embodiments of the present invention are based on the idea to not only detect a bloodstream obstructing object in a vessel structure for blood supply of at least one organ of a patient, but also to determine a perfusion defect score for a target region, which is potentially affected by the obstructing object, depending on energy resolved CT imaging data.

According to an aspect of one or more example embodiments of the present invention, a computer-implemented method for imaging-based characterization of a perfusion defect in a vessel structure, in particular a blood vessel structure, for blood supply of at least one organ of a patient is provided. Therein, medical imaging data depicting the at least one organ and the vessel structure is received, for example from a medical imaging device or from a data storage device. The medical imaging data comprises or consists of energy resolved CT imaging data. A blood stream obstructing object, for example a blood clot, in the vessel structure is detected based on the medical imaging data. A perfusion defect score for a target region of the at least one organ, wherein the blood perfusion in the target region is potentially affected by the obstructing object, is determined depending on the energy resolved CT imaging data.

Unless stated otherwise, all steps of the computer-implemented method may be performed by a data processing system, which comprises at least one data processing device. In particular, the at least one data processing device is configured or adapted to perform the steps of the computer-implemented method. For this purpose, the at least one data processing device may for example store a computer program comprising instructions which, when executed by the at least one data processing device, cause the at least one data processing device to execute the computer-implemented method. The expressions “data processing system” and “at least one data processing device” may be used interchangeably, here and in the following. This holds also for respective expressions derived therefrom. In other words, claims for the system according to one or more example embodiments of the present invention can be improved with features described or claimed in the context of the methods and vice versa. In this case, the functional features of the method are embodied by objective units or modules of the system.

In case the at least one data processing device comprises two or more data processing devices, certain steps carried out by the at least one data processing device may also be understood such that different data processing devices carry out different steps or different parts of a step. In particular, it is not required that each data processing device carries out the steps completely. In other words, carrying out the steps may be distributed amongst the two or more data processing devices.

From each implementation of the computer-implemented method, a respective implementation of a method for imaging-based characterization of a perfusion defect in a vessel structure for blood supply of at least one organ of a patient, which is not purely computer-implemented, is obtained by including respective steps of generating the medical imaging data, for example by the medical imaging device.

The energy resolved CT imaging data may comprise contrast enhanced imaging data obtained with X-ray computed tomography (CT) using contrast agents. Contrast agents for X-ray CT are often iodine-based contrast agents. This is useful to highlight structures such as blood vessels that otherwise would be difficult to delineate from their surroundings. Using contrast material can also help to obtain functional information about tissues. Often, images are taken both with and without radiocontrast.

The energy resolved CT imaging data, also denoted as spectrally resolved CT imaging data, is denoted as such, since it comprises imaging data for different energies of the detected X-rays or X-ray quanta, respectively. This may for example be achieved by using different X-rays spectra, for example by using different X-ray filters, by using X-ray sources to provide X-rays with different energy spectra, such as in dual source CT scanners or by kV-switching of a single X-ray source, or by using energy-resolving X-ray detectors, in particular via photon counting CT, PCCT, where different energy thresholds may be used for counting the incident photons. The medical imaging data, for example the energy resolved CT imaging data, may comprise image data derived from energy resolved CT imaging data raw data such as one or more reconstructed CT image volumes, including for example a regular CT image volume, imaging data with material decomposition information, virtual monoenergetic imaging data, an iodine-enhanced CT image volume, virtual non-contrast imaging data, a dual-energy ratio (DER), CT image volume DER, and so forth.

The energy resolved CT imaging data allows to differentiate between different materials. This may for example be exploited by computing respective contrast agent maps, for example iodine maps, or dual energy ratio, DER maps, which may then be analyzed to determine the perfusion defect score.

Detecting the obstructing object comprises, in particular, determining the location of the obstructing object in the vessel structure or a region of the vessel structure, wherein the obstructing object is located. This information is, for example, used to identify the target region. The detection of the obstructing object may be done depending on the energy resolved CT imaging data as well.

Alternatively, the medical imaging data may comprise further imaging data and the obstructing object is detected depending on the further imaging data. For detecting the obstructing object, for example a known image analysis algorithm may be used.

If not stated otherwise, here and in the following, CT imaging data may for example comprise one or more three-dimensional CT reconstructions, also denoted as reconstructed CT image volumes.

The at least one organ may for example comprise the lungs of the patient or a part of the lungs including, in particular, a bronchial tree of the lungs and/or parenchymal tissue of the lungs. The at least one organ may for example also include the vessel structure. The vessel structure may for example comprise a structure or tree of pulmonary arteries of the lungs.

The perfusion defect score may be a final result of the computer-implemented method according to one or more example embodiments of the present invention. In particular, by determining the perfusion defect score, the perfusion defect in a vessel structure is characterized. Depending on the perfusion defect score, for example the severity or criticality of the obstructing object's effect may be evaluated.

The size and location of the obstructing object can vary, ranging from small and peripheral to large and central. Consequently, the effect of the obstructing object can be very minor but can also be significant and even life-threatening, since the obstructing object may reduce or completely cut off blood supply to the downstream lung tissue, leading to decreased lung perfusion. The perfusion defect score may therefore be used to differentiate between more or less severe obstructing objects.

In particular, the perfusion defect score may be regarded as being assigned or associated with the concrete obstructing object. In other words, determining the perfusion defect score may be regarded as classifying the obstructing object according to the severity of its effect on the perfusion of the at least one organ.

In some implementations, the described steps of the computer-implemented method according to one or more example embodiments of the present invention may be carried out twice or multiple times for different obstructing objects at different locations, respectively. In this case, the different obstructing objects maybe ranked and/or filtered according to their perfusion defect scores.

According to several embodiments, a segmentation dividing the at least one organ into a plurality of segments is generated based on the medical imaging data, wherein the plurality of segments is hierarchically classified according to their blood supply by the vessel structure. The target region is determined to comprise or consist of one or more target segments of the plurality of segments, whose blood perfusion is potentially affected by the obstructing object according to the hierarchical classification.

In such embodiments, it is exploited that the anatomy of human organs, in particular the lungs, is well-known and so is the relation between segments of the organs and the respective vessels supplying them with blood. Thus, by using the segmentation including the hierarchical classification, a particularly reliable characterization of the perfusion defect is achieved.

For example, a known algorithm for medical image segmentation may be used to generate the plurality of segments.

In case the at least one organ comprises the lungs, the segmentation may for example be generated according to a bronchopulmonary segment model or according to a lung segmentation model, for example on the basis of the hierarchical classification according to Boyden mentioned in the introductory part of the present disclosure. The hierarchical classification of the plurality of segments according to their blood supply by the vessel structure may for example be understood such that it is defined for each segment of the plurality of segments, whether blood supply of the respective segment by the vessel structure depends on the blood supply of one or more other segments of the plurality of segments via the vessel structure and, if applicable, which other segments of the plurality of segments. In other words, given a segment of the plurality of segments and assuming that its blood supply via the vessel structure is completely blocked due to an obstructing object in the respective part of the vessel structure, it is defined to which other segments of the plurality of segments blood supply via the vessel structure would therefore also be cut off.

For example, by detecting the obstructing object, a root segment of the plurality of segments may be identified, which is the segment of the plurality of segments, where the obstructing object is located in the respective part of the vessel structure, which directly supplies the respective segment. In other words, the root segment is the segment of the plurality of segments, whose blood supply would be affected by the obstructing object, even if the blood supply of no other segment, in particular a hierarchically higher ordered segment, would be affected by the obstructing object. The root segment may also be determined as the segment of the plurality of segments to which the obstructing object lies closest.

The one or more target segments comprise the root segment. In some cases, the one or more target segments consist of the root segment. Alternatively, the one or more target segments comprises one or more downstream segments of the plurality of segments. In particular, the one or more target segments consist of the root segment and the one or more downstream segments. The one or more downstream segments may be those segments, whose blood supply via the vessel structure would be cut off in case the blood supply of the root segment was cut off.

According to several embodiments, for each of the one or more target segments, a respective segment perfusion defect score is determined based on the energy resolved CT imaging data and the perfusion defect score is determined depending on the segment perfusion defect scores, in particular depending on all segment perfusion defect scores of all target segments.

By evaluating the effect of the obstructing object individually for each target segment, a comprehensive analysis of the total perfusion defect is achieved and therefore a particularly reliable characterization of the perfusion defect.

For example, the perfusion defect score may be computed depending on or as a sum of the segment perfusion defect scores of the target segments or an average of the segment perfusion defect scores of the target segments or a maximum segment perfusion defect score of the target segments, et cetera. The perfusion defect score may also be computed as the number of target segments, whose segment perfusion defect score is at least a predefined threshold value.

According to several embodiments, the perfusion blood volume, PBV, may be determined for each of the target segments depending on the energy resolved CT imaging data and the segment perfusion defect scores may be computed depending on the respective PBV.

The PBV allows to reliably quantify the blood perfusion in the target segments and therefore to reliably quantify the effect of the obstructing object on the blood perfusion in the target segments. In particular, the PBV may be determined based on the quantification of iodine uptake of a contrast agent, for example iodine, by the organ, in particular lung parenchyma, based on the energy resolved CT imaging data. For example, the lower the PBV is and/or the larger the region of reduced PBV is, the larger is the effect of the obstructing object on the blood perfusion of the respective target segment and, consequently, the larger is the respective segment perfusion defect score, for example.

According to several embodiments, for each of the one or more target segments, a size of a perfusion defect region, for example a volume of the perfusion defect region, in the respective target segment is determined based on the energy resolved CT imaging data. The respective segment perfusion defect score is determined depending on or as the size of the perfusion defect region.

The size of the perfusion defect region may for example be determined as a volume in the respective target segment, where the PBV is less than a predefined threshold, for example.

The size of the perfusion defect region represents a reliable measure for assessing the effect of the obstructing object on the blood perfusion of the respective target segment. Thus, the segment perfusion defect scores and, consequently, perfusion defect score, may be determined in a particularly reliable way.

For example, the perfusion defect score may be computed as a total perfusion defect size or, in other words, total perfusion defect volume, which is given by a sum of the sizes of the perfusion defect regions of the individual target segments. The perfusion defect score may also be computed as a ratio of the total perfusion defect size to a predetermined or predefined total lung volume of the patient.

According to several embodiments, at least one PBV value for the target region is determined depending on the energy resolved CT imaging data and the perfusion defect score is determined depending on the at least one PBV value.

The at least one PBV value may for example be a single PBV value for the whole target region or a respective segment PBV value for each target segment.

A PBV value may be a value derived from the PBV of the target region or the respective target segment. For example, the at least one PBV value may correspond to the total perfusion defect size or the at least one PBV value comprises a respective segment PBV value for each of the one or more target segments, for example the respective size of the perfusion defect region of the respective target segment.

According to several embodiments, the medical imaging data, in particular the energy resolved CT imaging data, comprises photon-counting CT, PCCT, imaging data and the obstructing object is detected based on the PCCT imaging data.

Such embodiments are particularly beneficial, since on the one hand, PCCT allows for ultra-high resolution CT reconstructions and, on the other hand, also naturally may provide the data in energy resolved form by evaluating different energy thresholds when counting the incident X-ray photons. Thus, the obstructing object may be detected in a particularly accurate way and also the perfusion defect score may be obtained in a particularly reliable way.

Photon-counting detectors for computed tomography enable the generation of spectrally resolved computed tomography medical image data. A photon-counting detector can be con-figured to acquire x-ray projection data in a plurality on energy bins. These window parameters of the energy bins are defined by energy thresholds corresponding to the (spectral) energy of the X-ray photons to be detected. The energy thresholds of the detector can be predetermined and set by a respective control logic which can be implemented in the photon counting detector. A plurality of energy bins, for example four, can be used to cover the range of energies of the acquired x-ray projection data.

The window parameters of the energy bins may be pre-defined before the start of the acquisition of the energy resolved imaging raw data. To improve the quality of the acquired photon-counting spectral computed tomography data, the window parameters of the energy bins may be adapted with regard to the specific computed tomography application. For example, the window parameters of the energy bins can be preselected to obtain energy resolved imaging data that allows for optimized material decomposition of the energy resolved imaging, for example for any of the materials selected from the group consisting of iodine, calcium and water. In particular, it can be advantageous to provide three or more energy bins to enable a three-material decomposition. Thereby it is possible to obtain the energy resolved imaging data which is optimized for the task of detecting a perfusion defect or a vessel obstruction.

In embodiments, where the segmentation is generated based on the energy resolved CT imaging data, the segmentation may for example be generated based on the PCCT imaging data.

According to several embodiments, the segmentation is generated by applying a first trained machine learning model, MLM, to first input data comprising the medical imaging data at least partially, for example the energy resolved CT imaging data.

In general terms, a trained MLM may mimic cognitive functions that humans associate with other human minds. In particular, by training based on training data the MLM may be able to adapt to new circumstances and to detect and extrapolate patterns. Another term for a trained MLM is “trained function”.

For example, a trained MLM can be trained with energy resolved imaging data as training data, wherein the training data has been annotated with additional training information. The additional training information can comprise the annotation of ground truth information. As a non-limiting list of examples, the ground truth information can comprise a classification information which classifies a target region of the imaging data into one of a plurality of classes, a scoring information, which associates a target region of the image with a score (g.g.a disease score), an image segmentation information, a perfusion information, an indication of the presence or absence an obstruction of a vessel in a target region of the imaging data, or an indication of the presence or absence of a perfusion defect in a target region. The annotation can be performed manually by trained personnel and/or automatically by use or with the aid of a respective annotation software.

In general, parameters of an MLM can be adapted or updated via training. In particular, supervised training, semi-supervised training, unsupervised training, reinforcement learning and/or active learning can be used. Furthermore, representation learning, also denoted as feature learning, can be used. In particular, the parameters of the MLMs can be adapted iteratively by several steps of training. In particular, within the training a certain loss function, also denoted as cost function, can be minimized. In particular, within the training of an artificial neural network, ANN, the backpropagation algorithm can be used.

In particular, an MLM can comprise an ANN, a support vector machine, a decision tree and/or a Bayesian network, and/or the MLM can be based on k-means clustering, Q-learning, genetic algorithms and/or association rules. In particular, an ANN can be or comprise a deep neural network, a convolutional neural network or a convolutional deep neural network. Furthermore, an ANN can be an adversarial network, a deep adversarial network and/or a generative adversarial network, GAN.

The first MLM may be a known MLM for medical image segmentation, for example an ANN based on the U-Net, which has been trained in a conventional manner.

According to several embodiments, the obstructing object is detected by applying a second trained MLM to second input data comprising the medical imaging data at least partially, for example the energy resolved CT imaging data.

The second MLM may be a known MLM for medical image segmentation or object detection in medical images, for example an ANN based on the U-Net or an algorithm as proposed by Grenier et al. in the above mentioned publication, which has been trained in a conventional manner.

In some embodiments, the first MLM and the second MLM may also be part of a further MLM. In such embodiments, the obstructing object is detected and the segmentation is generated by applying the trained further MLM to at least a part of the medical imaging data, for example of the energy resolved CT imaging data.

According to several embodiments, the energy resolved CT imaging data comprises contrast enhanced CT, CECT, imaging data, for example including a contrast agent map, in particular an iodine map, and/or a DER map.

Consequently, in such embodiments, the perfusion defect score, in particular the segment perfusion defect scores and/or the size of perfusion defect regions and/or the PBV values, may be determined with an increased accuracy.

According to several embodiments, the perfusion defect score is determined by applying a trained third MLM to third input data comprising the medical imaging data.

The third MLM may for example be based on a known MLM architecture, for example the U-Net, which has been trained in a conventional manner. In particular, the training of the third MLM may be based on a plurality of training datasets, each of them comprising respective training imaging data corresponding to the medical imaging data and a corresponding perfusion defect score as a ground truth label.

Consequently, the trained third MLM can directly predict the perfusion defect score. In such embodiments, carrying out the computer-implemented method according to the present invention is particularly efficient from a computational point of view.

For example, an output of the third MLM as a result of applying it to the third input data may comprise a location of the obstructing object as well. In this case, the training data sets may also comprise a corresponding location as a ground truth label.

According to several embodiments, the third input data comprises the segmentation including the hierarchical classification.

In the respective training phase, the training data sets also comprise a corresponding training segmentation as an input to the third MLM. In such embodiments, the training effort for the third MLM may be reduced, since the third MLM is given additional information about the content of the training imaging data. Furthermore, also the accuracy of the trained third MLM's output may be increased.

According to a further aspect of one or more example embodiments of the present invention, a data processing system is provided. The data processing system is configured to carry out a computer-implemented method according to one or more example embodiments of the present invention.

In the present disclosure, the expressions “data processing system” and “at least one data processing device” may be used interchangeably. A data processing device may in particular be understood as a data processing device, which comprises processing circuitry. The data processing device can therefore in particular process data to perform computing operations. This may also include operations to perform indexed accesses to a data structure, for example a look-up table, LUT, as well as a data processing process implemented in hardware.

In particular, the data processing device may include one or more computers, one or more microcontrollers, and/or one or more integrated circuits, for example, one or more application-specific integrated circuits, ASIC, one or more field-programmable gate arrays, FPGA, and/or one or more systems on a chip, SoC. The data processing device may also include one or more processors, for example one or more microprocessors, one or more central processing units, CPU, one or more graphics processing units, GPU, and/or one or more signal processors, in particular one or more digital signal processors, DSP. The data processing device may also include a physical or a virtual cluster of computers or other of said units.

In various embodiments, the data processing device includes one or more hardware and/or software interfaces and/or one or more memory units.

A memory unit may be implemented as a volatile data memory, for example a dynamic random access memory, DRAM, or a static random access memory, SRAM, or as a non-volatile data memory, for example a read-only memory, ROM, a programmable read-only memory, PROM, an erasable programmable read-only memory, EPROM, an electrically erasable programmable read-only memory, EEPROM, a flash memory or flash EEPROM, a ferroelectric random access memory, FRAM, a magnetoresistive random access memory, MRAM, or a phase-change random access memory, PCRAM.

In particular, the data processing system may comprise an input data interface, configured to receive medical imaging data depicting a vessel structure in at least one organ and the vessel structure is received, wherein the medical imaging data,) comprises energy resolved CT imaging data,
a detection module for detecting a blood stream obstructing object in the vessel structure is based on the medical imaging data; and
determination module for determining a perfusion defect score for a target region of the at least one organ, whose blood perfusion is potentially affected by the obstructing object, wherein said determining is performed depending on the energy resolved CT imaging data.

According to a further aspect of one or more example embodiments of the present invention, a medical imaging system is provided. The medical imaging system comprises a data processing system according to one or more example embodiments of the present invention and a CT device, which is configured to generate the medical imaging data depicting the at least one organ of the vessel structure.

In particular, the CT device is configured to generate the energy resolved CT imaging data. For example, the CT device may be a photon counting CT device.

Further implementations of the medical imaging system according to one or more example embodiments of the present invention follow directly from the various embodiments of the computer-implemented method according to the present invention and vice versa. In particular, individual features and corresponding explanations as well as advantages relating to the various implementations of the computer-implemented method according to one or more example embodiments of the present invention can be transferred analogously to corresponding implementations of the medical imaging system according to one or more example embodiments of the present invention. In particular, the medical imaging system according to one or more example embodiments of the present invention is designed or programmed to carry out the computer-implemented method according to one or more example embodiments of the present invention. In particular, the medical imaging system according to one or more example embodiments of the present invention carries out the computer-implemented method according to one or more example embodiments of the present invention.

According to a further aspect of one or more example embodiments of the present invention, a computer program comprising instructions is provided. When the instructions are executed by a data processing system, the instructions cause the data processing system to carry out a computer-implemented method according to one or more example embodiments of the present invention.

The instructions may be provided as program code, for example.

The program code can for example be provided as binary code or assembler and/or as source code of a programming language, for example C, and/or as program script, for example Python.

According to a further aspect, a non-transitory computer-readable storage medium storing a computer program according to one or more example embodiments of the present invention is provided.

The computer program and the computer-readable storage medium are respective computer program products comprising the instructions.

Further features and feature combinations of one or more example embodiments of the present invention are obtained from the figures and their description as well as the claims. In particular, further implementations of one or more example embodiments of the present invention may not necessarily contain all features of one of the claims. Further implementations of one or more example embodiments of the present invention may comprise features or combinations of features, which are not recited in the claims.

### BRIEF DESCRIPTION OF THE DRAWINGS

In the following, the present invention will be explained in detail with reference to specific exemplary embodiments and respective schematic drawings. In the drawings, identical or functionally identical elements may be denoted by the same reference signs. The description of identical or functionally identical elements is not necessarily repeated with respect to different figures.

In the figures,

FIG. 1 shows an exemplary embodiment of a medical imaging system according to the present invention;

FIG. 2 shows a schematic flow diagram of an exemplary embodiment of a computer-implemented method for imaging-based characterization of a perfusion defect in a vessel structure for blood supply of at least one organ of a patient according to the present invention;

FIG. 3 shows schematically a bronchial tree and an obstructing object in a use case for a further exemplary embodiment of a computer-implemented method for imaging-based characterization of a perfusion defect according to the present invention;

FIG. 4 shows schematically a bronchial tree and an obstructing object in a use case for a further exemplary embodiment of a computer-implemented method for imaging-based characterization of a perfusion defect according to the present invention;

FIG. 5 shows schematically a transversal sectional view of segmented lungs in a use case for a further exemplary embodiment of a computer-implemented method for imaging-based characterization of a perfusion defect according to the present invention;

FIG. 6 shows schematically a sagittal sectional view of segmented lungs in a use case for a further exemplary embodiment of a computer-implemented method for imaging-based characterization of a perfusion defect according to the present invention;

FIG. 7 shows a schematic flow diagram of a further exemplary embodiment of a computer-implemented method for imaging-based characterization according to the present invention;

FIG. 8 shows a schematic flow diagram of a further exemplary embodiment of a computer-implemented method for imaging-based characterization according to the present invention;

FIG. 9 shows schematically an exemplary embodiment of an artificial neural network;

FIG. 10 shows schematically an exemplary embodiment of a convolutional neural network; and

FIG. 11 shows schematically a further exemplary embodiment of a convolutional neural network.

### DETAILED DESCRIPTION

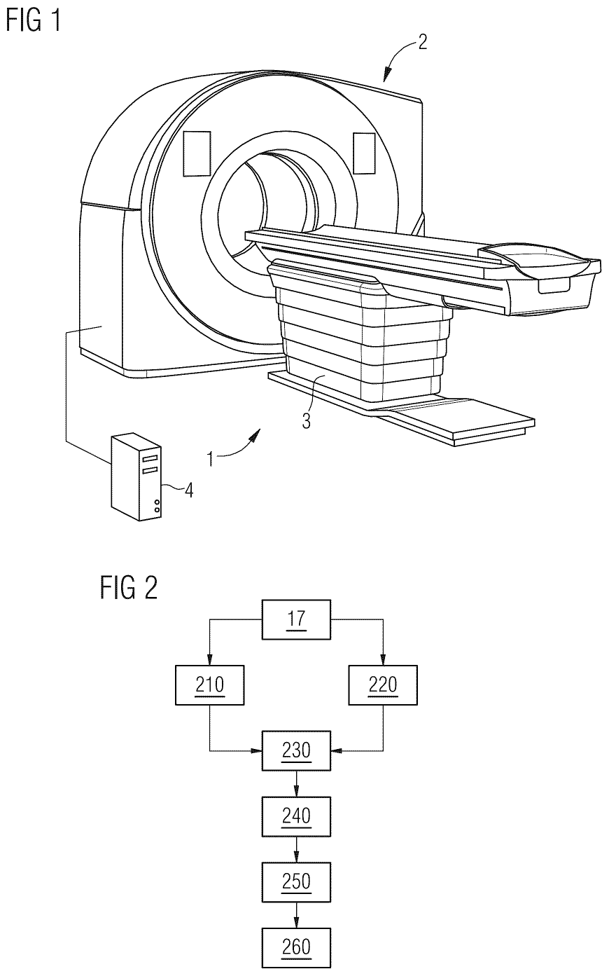

FIG. 1 shows an exemplary embodiment of a medical imaging system 1 according to the present invention. The medical imaging system 1 comprises a data processing system 4 according to one or more example embodiments of the present invention and a CT device 2, which is, in particular, implemented as a photon-counting CT device.

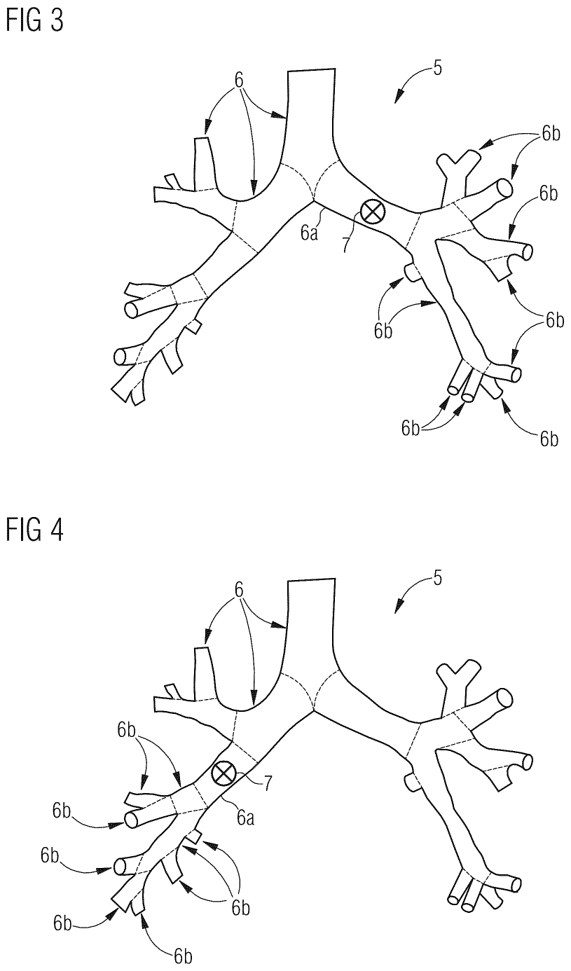

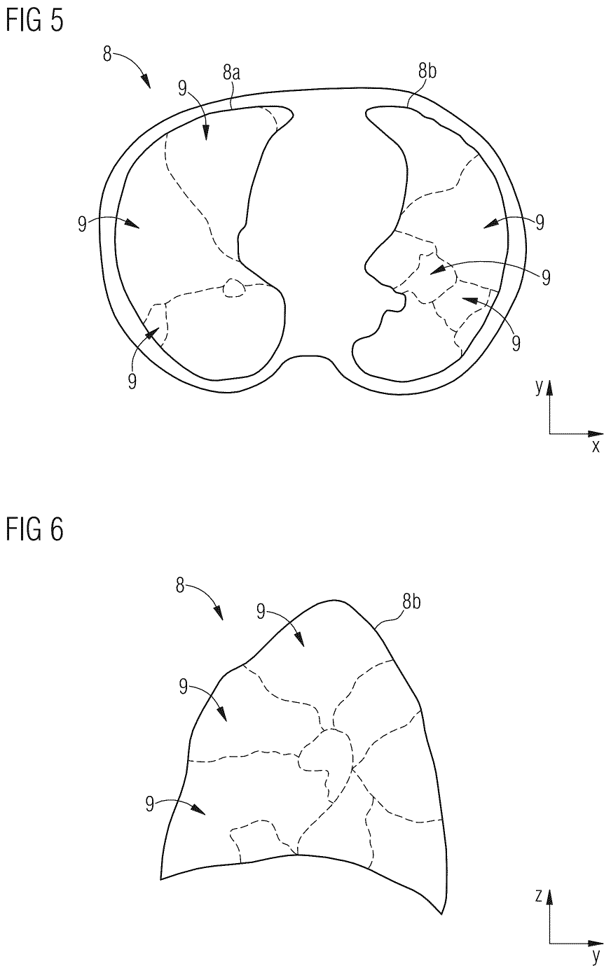

The CT device 2 is configured to generate energy resolved CT imaging data 17, in particular including one or more reconstructed CT image volumes, depicting at least one organ 5, 8 of a patient and a vessel structure for blood supply of at least one organ 5, 8. The at least one organ 5, 8 is, in particular, given by lungs 8 of the patient including a bronchial tree 5, as shown schematically in FIG. 3 and FIG. 4, and parenchymal tissue of the lungs 8, as shown schematically in FIG. 5 and FIG. 6. The vessel structure may for example include or correspond to pulmonary arteries.

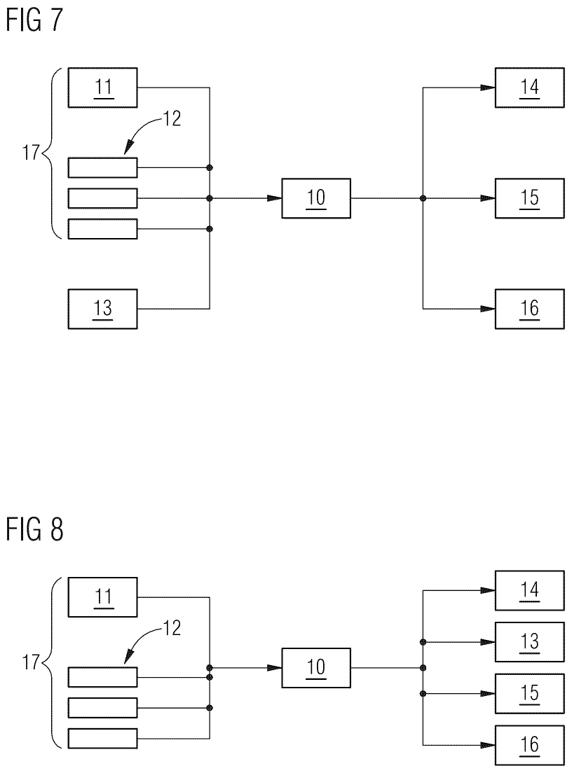

The data processing system 4 is configured to carry out an exemplary embodiment of a computer-implemented method for imaging-based characterization of a perfusion defect in the vessel structure according to the present invention. Schematic flow diagrams of different exemplary embodiments of said computer-implemented method are shown in FIG. 2, FIG. 7 and FIG. 8.

In general, in order to carry out a computer-implemented method according to one or more example embodiments of the present invention, the data processing system 4 receives the energy resolved CT imaging data 17 and detects a blood stream obstructing object 7, in particular a blood clot, in the vessel structure based on the energy resolved CT imaging data 17. The data processing system 4 determines a perfusion defect score 16 for a target region 6a, 6b of the at least one organ 5, 8, wherein the blood perfusion of the target region 6a, 6b, is potentially affected by the obstructing object 7, depending on the energy resolved CT imaging data 17.

According to the embodiment of the computer-implemented method depicted in FIG. 2, the obstructing object 7 is detected in step 210 using for example a known algorithm, for example an algorithm based on a trained first MLM, for clot detection in pulmonary arteries. Beyond the known method, the detection of the obstructing object 7 may for example be improved by using spectral information of the energy resolved CT imaging data 17, for example a material-labeling to better differentiate between contrast agent, in particular, iodine, and clot material.

In step 220, a segmentation 13 dividing the at least one organ 5, 8 into a plurality of segments 6, 9 is generated based on the medical imaging data 17, wherein the plurality of segments 6, 9 is hierarchically classified according to their blood supply by the vessel structure.

The segmentation may for example be a segmentation of the bronchial tree 5 as shown in FIG. 3 and FIG. 4. The segmentation may for example be based on the classification by Boyden. The trachea divides into two main bronchi, also known as primary bronchi, at the carina. One main bronchus leads to the right lung 8b, and the other leads to the left lung 8a. Each main bronchus enters the respective lung and further divides. The main bronchi divide into lobar bronchi, also called secondary bronchi. The right lung 8b has three lobes, so the right main bronchus divides into three lobar bronchi, denoted as superior, middle, and inferior lobar bronchi. The left lung 8a has two lobes, so the left main bronchus divides into two lobar bronchi, namely superior and inferior lobar bronchi. The lobar bronchi further divide into segmental bronchi. The segmental bronchi supply specific segments of lung tissue known as bronchopulmonary segments. Each lung has a varying number of bronchopulmonary segments, with the right lung 8b having more bronchopulmonary segments compared to the left lung 8a. The segmental bronchi continue to divide into smaller bronchi known as subsegmental bronchi. These subsegmental bronchi supply even smaller regions within the bronchopulmonary segments.

The bronchial tree 5 is not only responsible for the passage of air but also requires its own blood supply by the pulmonary arteries. The pulmonary arteries branch off from the systemic circulation and supply oxygenated blood to the bronchi, bronchioles, and other lung structures. Each segment 6 of the bronchial tree 5 has its own pulmonary arterial branch and thus, each lung segment 9, as shown FIG. 5 and FIG. 6, is a portion of lung supplied by its own bronchus and artery. Each segment is functionally and anatomically discrete allowing a single segment to be surgically resected without affecting its neighboring segments. Thus, each segment 6 of the bronchial tree 5 can also be associated to a respective segment 9 of the parenchymal tissue of the lungs 8 and, in particular, an obstruction of the blood flow in a part of the vessel structure supplying a certain segment 6 of the bronchial tree 5 potentially affects the associated blood perfusion of the corresponding segment 9 of the parenchymal tissue of the lungs 8.

The classification of bronchi according to Boyden refers to the standard nomenclature used to describe bronchopulmonary segmental anatomy. The described bronchopulmonary segment model is a preferred segmentation. However, the segmentation may also be based on another segment model, for example of the lung lobes, left and right lung 8b, or other customized lung segment definitions.

In the method of FIG. 2, the target region 6a, 6b is determined to comprise one or more target segments 6a, 6b of the plurality of segments 6, 9, whose blood perfusion is potentially affected by the obstructing object 7 according to the hierarchical classification. It is noted that the location of the obstructing object 7 is shown in FIG. 3 and FIG. 4 in certain segments 6a of the bronchial tree 5 for illustrative reasons. This does not mean that the object 7 is located inside the respective bronchus but rather in the corresponding part of the vessel structure.

As shown in FIG. 5 and FIG. 6, the parenchymal tissue of the lungs 8 may be segmented based on a known algorithm, for example based on a trained second MLM. For example, the second MLM may be trained on ultra-high-resolution photon-counting CT data that better depicts the intersegmental fissures allowing for a better anatomical delineation of the lung segments.

In step 230, the root segment 6a of the bronchial segments 6, which is the segment 6 corresponding to the location of the obstructing object 7, and/or the corresponding root segment of the parenchymal tissue of the lungs 8 is automatically identified based on the segmentation 13.

Based on the hierarchical anatomical bronchopulmonary segment model, all dependent downstream segments 6b of the bronchial tree 5 and/or all dependent downstream segments of the parenchymal tissue of the lungs 8 are identified in step 240.

In each of the downstream segments of the parenchymal tissue of the lungs 8, the parenchymal perfusion is quantified in step 250 based on spectral perfusion data obtained from the energy resolved CT imaging data 17, for example based on the iodine uptake or dual energy ratio DER. For example, the spectral perfusion data may be normalized based on reference values in an automatically identified reference region, for example the pulmonary trunk, to account for contrast bolus variations in-between different scans and patients. The quantification may for example be done by comparing the iodine concentration, enhancement values or DER between the downstream segments of the parenchymal tissue of the lungs 8 and normal lung segments. The normal lung segments may for example be defined as the perfusion in a normal reference population or as the perfusion in non-affected downstream segments in a given patient.

In step 260, the perfusion defect score 16 is then aggregated from the segment-wise perfusion defect quantification. The perfusion defect score 16 may for example be determined as the number of segments with perfusion defects or significant perfusion defects, a total volume of the perfusion defect regions, a percentage of lung involvement calculated by dividing the volume of the perfusion defects by the total lung volume, et cetera.

The steps 210 to 260 may also be carried out repeatedly for different associated obstructing objects 7, see FIG. 3 in comparison to FIG. 4, for example. In particular, the associated obstructing objects 7 may be ranked or filtered according to their severeness given by their perfusion defect score 16. Also, in some embodiments, perfusion defects which could not be explained by associated obstructing objects 7 may be marked for further analysis.

FIG. 7 shows a schematic flow diagram of a further exemplary embodiment of a computer-implemented method for imaging-based characterization according to the present invention. Therein, the perfusion defect score 16 is determined by applying a trained third MLM 10 to third input data comprising the energy resolved CT imaging data 17. In particular, in the embodiment of FIG. 7, the detection of the location 14 of the obstructing object 7 or of multiple such obstructing objects 7 and associated perfusion defect scores 16 is combined intrinsically into the third MLM 10, which may be based on a U-Net or a transformer model, for example.

The third MLM 10 receives as an input, for example, a CECT image volume 11, preferably with ultra-high resolution, and spectral information 12, such as the associated iodine image or DER-image. Optionally, the segmentation 13 with hierarchical labeling may be passed to the third MLM 10 as an additional input to guide the third MLM 10 towards adhering to given lung segments. If the lung segments are not passed as input, the third MLM 10 may for example identify perfusion defect regions not adhering to any prior segment model.

The output of the third MLM 10 includes, for example, the respective locations of the detected obstructing objects 7.

Additionally, detection algorithm may also be trained on the spectral information 12 that helps to differentiate blood clot material from iodine, for example. The output of the third MLM 10 also includes the respective perfusion defect scores 16. Optionally, the output of the third MLM 10 includes respective detected regions with perfusion defects and corresponding perfusion defect quantifications for each obstructing objects.

FIG. 8 shows a schematic flow diagram of a further exemplary embodiment of a computer-implemented method for imaging-based characterization according to the present invention. As explained for FIG. 7, the perfusion defect score 16 is determined by applying the trained third MLM 10 to the third input data comprising the energy resolved CT imaging data 17. The input for the third MLM 10 includes, for example, the CECT image volume 11 and the spectral information 12, but not the segmentation 13. Rather, in this embodiment the third MLM is trained on an auxiliary task predict the segmentation 13 as part of its output.

The third MLM 10 for the embodiments of FIG. 7 and FIG. 8 above are for example trained in a supervised manner based on expert annotations for the output data to be estimated.

In several embodiments of the computer-implemented method according to the present invention, clot detection and parenchymal perfusion defect assessment are combined to improve the detection of severe pulmonary embolism thrombi with associated perfusion defects.

In several embodiments, PCCT imaging data is used as a basis leveraging the intrinsic benefits of ultra-high-resolution and spectral image information provided by PCCT scans. This helps to separate pulmonary embolisms that do not have severe impact to the patient from more critical ones. For example, a blood clot may have a hyperdense appearance, which is difficult to differentiate on contrast-enhanced conventional CT images. However, blood clots and iodine have distinct material compositions that can be assessed by spectral PCCT more accurately.

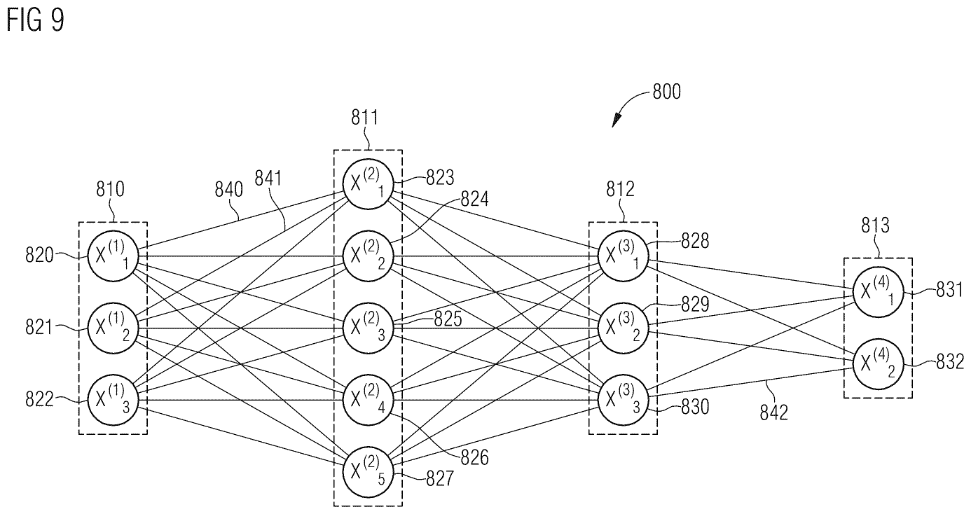

As explained above, several method steps may be carried out using respectively trained MLMs, for example ANNs. FIG. 9 displays an embodiment of an ANN 800, which is for example designed as an MLP. The ANN 800 comprises nodes 820, . . . , 832 and edges 840, . . . , 842, wherein each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832. It is, however, also possible that the first node 820, . . . , 832 and the second node 820, . . . , 832 are identical. For example, in FIG. 9, the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as ingoing edge for the second node 820, . . . , 832 and as outgoing edge for the first node 820, . . . , 832.

In this example, the nodes 820, . . . , 832 of the artificial neural network 800 can be arranged in layers 810, . . . , 813, wherein the layers can comprise an intrinsic order introduced by the edges 840, . . . , 842 between the nodes 820, . . . , 832. In particular, edges 840, . . . , 842 can exist only between neighboring layers of nodes. In the displayed example, there is an input layer 810 comprising only nodes 820, . . . , 822 without an incoming edge, an output layer 813 comprising only nodes 831, 832 without outgoing edges, and hidden layers 811, 812 in-between the input layer 810 and the output layer 813. In general, the number of hidden layers 811, 812 can be chosen arbitrarily. In an MLP, this number is at least one. The number of nodes 820, . . . , 822 within the input layer 810 usually relates to the number of input values of the artificial neural network 800, and the number of nodes 831, 832 within the output layer 813 usually relates to the number of output values of the artificial neural network 800.

In particular, a real number can be assigned as a value to every node 820, . . . , 832 of the artificial neural network 800. Here, x(n)i denotes the value of the i-th node 820, . . . , 832 of the n-th layer 810, . . . , 813. The values of the nodes 820, . . . , 822 of the input layer 810 are equivalent to the input values of the artificial neural network 800. The values of the nodes 831, 832 of the output layer 813 are equivalent to the output value of the artificial neural network 800. Furthermore, each edge 840, . . . , 842 can comprise a weight being a real number. In particular, the weight is a real number within the interval [−1, 1] or within the interval [0, 1]. Here, w(m,n)i,j denotes the weight of the edge between the i-th node 820, . . . , 832 of the m-th layer 810, . . . , 813 and the j-th node 820, . . . , 832 of the n-th layer 810, . . . , 813. Furthermore, the abbreviation w(n)i,j is defined for the weight w(n,n+1)i,j. In particular, to calculate the output values of the neural network 800, the input values are propagated through the neural network 800. In particular, the values of the nodes 820, . . . , 832 of the (n+1)-th layer 810, . . . , 813 can be calculated based on the values of the nodes 820, . . . , 832 of the n-th layer 810, . . . , 813 by

$${x}_{j}^{( n + 1 )} = f ( {\sum }_{i} {x}_{i}^{( n )} {w}_{i , j}^{( n )} ) .$$

Herein, the function f is denoted as transfer function or activation function. Known transfer functions are step functions, the sigmoid functions, for example the logistic function, the generalized logistic function, the hyperbolic tangent, the arctangent function, the error function, the smoothstep function, or rectifier functions. The transfer function is for example used for normalization purposes. In particular, the values are propagated layer-wise through the neural network 800, wherein values of the input layer 810 are given by the input of the neural network 800, wherein values of the first hidden layer 811 can be calculated based on the values of the input layer 810 of the neural network 800, wherein values of the second hidden layer 812 can be calculated based on the values of the first hidden layer 811, and so forth.

In order to set the values w(m,n)i,j for the edges, the neural network 800 has to be trained using training data. In particular, training data comprises training input data and training output data (denoted as ti). For a training step, the neural network 800 is applied to the training input data to generate calculated output data. In particular, the training data and the calculated output data comprise a number of values, said number being equal with the number of nodes of the output layer. In particular, a comparison between the calculated output data and the training data is used to recursively adapt the weights within the neural network 800 (backpropagation algorithm). In particular, the weights are changed according to

$${w}_{i , j}^{′ ( n )} = {w}_{i , j}^{( n )} - γ {δ}_{j}^{( n )} {x}_{i}^{( n )} ,$$

wherein γ is a predefined learning rate, and the numbers δ(n)j can be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

based on δ(n+1)j, if the (n+1)-th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)-th layer is the output layer 813, wherein f′ is the first derivative of the activation function, and t(n+1)j is the comparison training value for the j-th node of the output layer 813.

The ANN may for example be a convolutional neural network, CNN. A CNN is an ANN that uses a convolution operation instead of general matrix multiplication in at least one of its layers. These layers are denoted as convolutional layers. In particular, a convolutional layer performs a dot product of one or more convolution kernels with the convolutional layer's input data, wherein the entries of the one or more convolution kernel are parameters or weights that may be adapted by training. In particular, one can use the Frobenius inner product and the ReLU activation function. A convolutional neural network can comprise additional layers, for example pooling layers, fully connected layers, and/or normalization layers.

By using convolutional neural networks, the input can be processed in a very efficient way because a convolution operation based on different kernels can extract various image features so that by adapting the weights of the convolution kernel the relevant image features can be found during training. Furthermore, based on the weight-sharing in the convolutional kernels fewer parameters need to be trained, which prevents overfitting in the training phase and allows to have faster training or more layers in the network, improving the performance of the network.

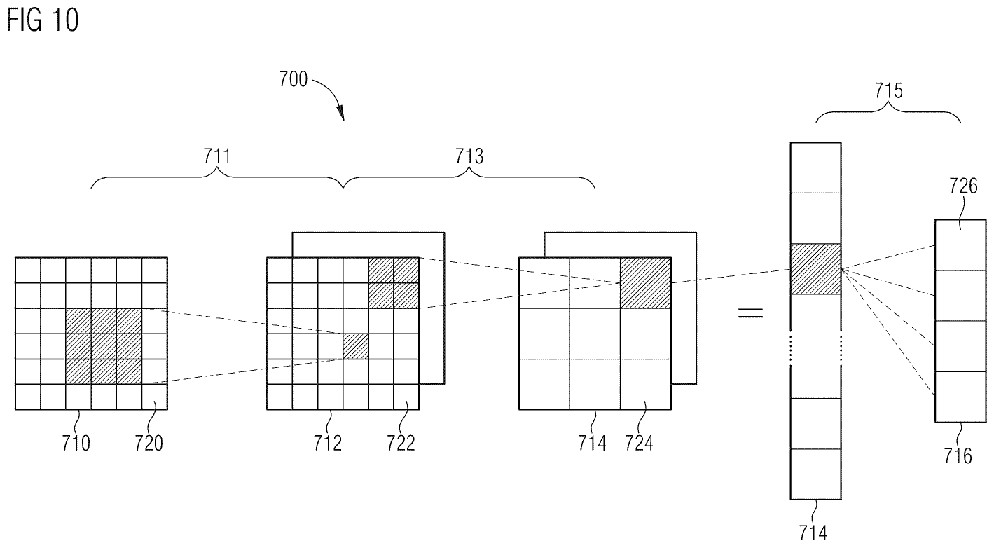

FIG. 10 displays an exemplary embodiment of a convolutional neural network 700. In the displayed embodiment, the convolutional neural network 700 comprises an input node layer 710, a convolutional layer 711, a pooling layer 713, a fully connected layer 714 and an output node layer 716, as well as hidden node layers 712, 714.

Alternatively, the convolutional neural network 200 can comprise several convolutional layers 711, several pooling layers 713 and/or several fully connected layers 715, as well as other types of layers.

The order of the layers can be chosen arbitrarily, usually fully connected layers 715 are used as the last layers before the output layer 716.

In particular, within a convolutional neural network 700 nodes 720, 722, 724 of a node layer 710, 712, 714 can be considered to be arranged as a d-dimensional matrix or as a d-dimensional image. In particular, in the two-dimensional case the value of the node 720, 722, 724 indexed with i and j in the n-th node layer 710, 712, 714 can be denoted as x(n)[i, j]. However, the arrangement of the nodes 720, 722, 724 of one node layer 710, 712, 714 does not have an effect on the calculations executed within the convolutional neural network 700 as such, since these are given solely by the structure and the weights of the edges.

A convolutional layer 711 is a connection layer between an anterior node layer 710 with node values x(n−1) and a posterior node layer 712 with node values x(n). In particular, a convolutional layer 711 is characterized by the structure and the weights of the incoming edges forming a convolution operation based on a certain number of kernels. In particular, the structure and the weights of the edges of the convolutional layer 711 are chosen such that the values x(n) of the nodes 722 of the posterior node layer 712 are calculated as a convolution x(n)=K*x(n−1) based on the values x(n−1) of the nodes 720 anterior node layer 710, where the convolution * is defined in the two-dimensional case as

$${x}^{( n )} [ i , j ] = ( K * {x}^{( n - 1 )} ) [ i , j ] = {\sum }_{i ′} {\sum }_{j ′} K [ {i}^{′} , {j}^{′} ] {x}^{( n - 1 )} [ i - {i}^{′} , j - {j}^{′} ] .$$

Herein, the kernel K is a d-dimensional matrix, in the present example a two-dimensional matrix, which is usually small compared to the number of nodes 720, 722, for example a 3×3 matrix, or a 5×5 matrix. In particular, this implies that the weights of the edges in the convolution layer 711 are not independent, but chosen such that they produce said convolution equation. In particular, for a kernel being a 3×3 matrix, there are only 9 independent weights, each entry of the kernel matrix corresponding to one independent weight, irrespectively of the number of nodes 720, 722 in the anterior node layer 710 and the posterior node layer 712.

In general, convolutional neural networks 700 use node layers 710, 712, 714 with a plurality of channels, in particular, due to the use of a plurality of kernels in convolutional layers 711. In those cases, the node layers can be considered as (d+1)-dimensional matrices, the first dimension indexing the channels. The action of a convolutional layer 711 is then in a two-dimensional example defined as

$${x}_{b}^{( n )} [ i , j ] = {\sum }_{a} ( {K}_{a , b} * {x}_{a}^{( n - 1 )} [ i , j ] = {\sum }_{a} {\sum }_{i ′} {\sum }_{j ′} {K}_{a , b} [ {i}^{′} , {j}^{′} ] \cdot {x}_{a}^{( n - 1 )} [ i - {i}^{′} , j - {j}^{′} ] ,$$

wherein

$${x}_{a}^{( n )}$$

corresponds to the a-th channel of the anterior node layer 710

$${x}_{b}^{( n )}$$

corresponds to the b-th channel of the posterior node layer 712 and K_{a,b} corresponds to one of the kernels. If a convolutional layer 711 acts on an anterior node layer 710 with A channels and outputs a posterior node layer 712 with B channels, there are A·B independent d-dimensional kernels K_{a,b}.

In general, in convolutional neural networks 700 activation functions may be used. In this embodiment, ReLU (rectified linear unit) is used, with R(z)=max(0, z), so that the action of the convolutional layer 711 in the two-dimensional example is

$${x}_{b}^{( n )} [ i , j ] = R ( {\sum }_{a} ( {K}_{a , b} * {x}_{a}^{( n - 1 )} [ i , j ] ) = {\sum }_{a} {\sum }_{i ′} {\sum }_{j ′} {K}_{a , b} [ {i}^{′} , {j}^{′} ] \cdot {x}_{a}^{( n - 1 )} [ i - {i}^{′} , j - {j}^{′} ] ) .$$

It is also possible to use other activation functions, for example ELU (exponential linear unit), LeakyReLU, Sigmoid, Tanh or Softmax.

In the displayed embodiment, the input layer 710 comprises 36 nodes 720, arranged as a two-dimensional 6×6 matrix. The first hidden node layer 712 comprises 72 nodes 722, arranged as two two-dimensional 6×6 matrices, each of the two matrices being the result of a convolution of the values of the input layer with a 3×3 kernel within the convolutional layer 711. Equivalently, the nodes 722 of the first hidden node layer 712 can be interpreted as arranged as a three-dimensional 2×6×6 matrix, wherein the first dimension correspond to the channel dimension.

An advantage of using convolutional layers 711 is that spatially local correlation of the input data can exploited by enforcing a local connectivity pattern between nodes of adjacent layers, in particular by each node being connected to only a small region of the nodes of the preceding layer.

A pooling layer 713 is a connection layer between an anterior node layer 712 with node values x(n−1) and a posterior node layer 714 with node values x(n). In particular, a pooling layer 713 can be characterized by the structure and the weights of the edges and the activation function forming a pooling operation based on a non-linear pooling function f. For example, in the two-dimensional case the values x(n) of the nodes 724 of the posterior node layer 714 can be calculated based on the values x(n−1) of the nodes 722 of the anterior node layer 712 as

$${x}_{b}^{( n )} [ i , j ] = f ( {x}_{b}^{( n - 1 )} [ {id}_{1} , {id}_{2} ] , … , {x}_{b}^{( n - 1 )} [ ( i + 1 ) {d}_{1} - 1 , ( j + 1 ) {d}_{2} - 1 ] ) .$$

In other words, by using a pooling layer 713, the number of nodes 722, 724 can be reduced by re-placing a number d1·d2 of neighboring nodes 722 in the anterior node layer 712 with a single node 722 in the posterior node layer 714 being calculated as a function of the values of said number of neighboring nodes. In particular, the pooling function f can be the max-function, the average or the L2-Norm. In particular, for a pooling layer 713 the weights of the incoming edges are fixed and are not modified by training.

The advantage of using a pooling layer 713 is that the number of nodes 722, 724 and the number of parameters is reduced. This leads to the amount of computation in the network being reduced and to a control of overfitting.

In the displayed embodiment, the pooling layer 713 is a max-pooling layer, replacing four neighboring nodes with only one node, the value being the maximum of the values of the four neighboring nodes. The max-pooling is applied to each d-dimensional matrix of the previous layer. In this embodiment, the max-pooling is applied to each of the two two-dimensional matrices, reducing the number of nodes from 72 to 18.

In general, the last layers of a convolutional neural network 700 may be fully connected layers 715. A fully connected layer 715 is a connection layer between an anterior node layer 714 and a posterior node layer 716. A fully connected layer 713 can be characterized by the fact that a majority, in particular, all edges between nodes 714 of the anterior node layer 714 and the nodes 716 of the posterior node layer are present, and wherein the weight of each of these edges can be adjusted individually.

In this embodiment, the nodes 724 of the anterior node layer 714 of the fully connected layer 715 are displayed both as two-dimensional matrices, and additionally as non-related nodes, indicated as a line of nodes, wherein the number of nodes was reduced for a better presentability. This operation is also denoted as flattening. In this embodiment, the number of nodes 726 in the posterior node layer 716 of the fully connected layer 715 smaller than the number of nodes 724 in the anterior node layer 714. Alternatively, the number of nodes 726 can be equal or larger.

Furthermore, in this embodiment the Softmax activation function is used within the fully connected layer 715. By applying the Softmax function, the sum the values of all nodes 726 of the output layer 716 is 1, and all values of all nodes 726 of the output layer 716 are real numbers between 0 and 1. In particular, if using the convolutional neural network 700 for categorizing input data, the values of the output layer 716 can be interpreted as the probability of the input data falling into one of the different categories.

In particular, convolutional neural networks 700 can be trained based on the backpropagation algorithm. For preventing overfitting, methods of regularization can be used, for example dropout of nodes 720, . . . , 724, stochastic pooling, use of artificial data, weight decay based on the L1 or the L2 norm, or max norm constraints.

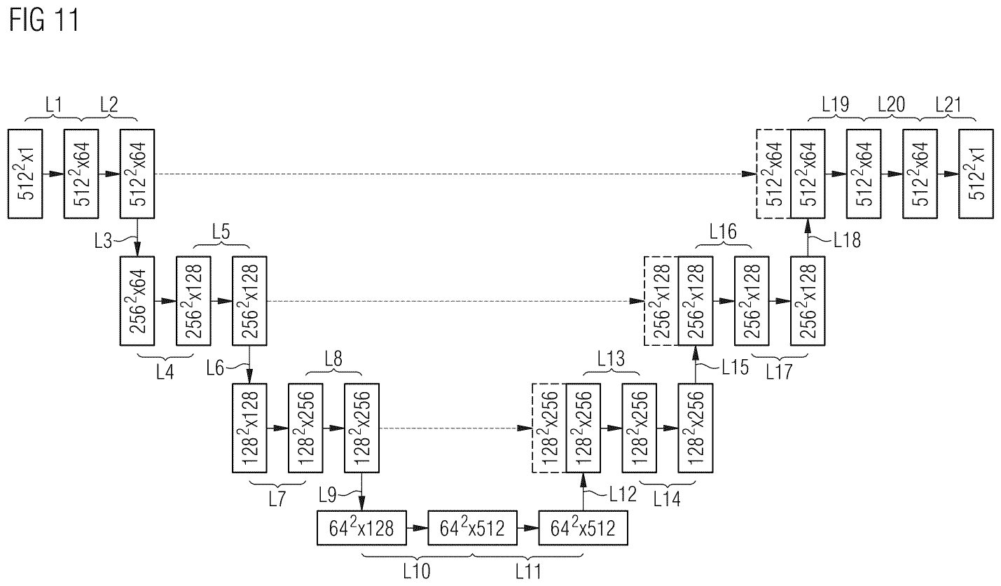

In the example of FIG. 11, the CNN has a U-Net structure. In the displayed example, the input data to the CNN is a two-dimensional medical image comprising 512×512 pixels, every pixel comprising one intensity value. The CNN comprises convolutional layers indicated by solid, horizontal arrows, pooling layers indicating by solid arrows pointing down, and upsampling layers indicated by solid arrows pointing up. The number of the respective nodes is indicated within the boxes. Within the U-Net structure first the input images are downsampled, in particular by decreasing the size of the images and increasing the number of channels. Afterwards they are upsampled, in particular by increasing the size of the images and decreasing the number of channels, to generate a transformed image.

All except the last convolutional layers L1, L2, L4, L5, L.7, L8, L10, L11, L13, L14, L16, L17, L19, L20 use 3×3 kernels with a padding of 1, the ReLU activation function, and a number of filters or convolutional kernels that matches the number of channels of the respective node layers as indicated in FIG. 11. The last convolutional layer uses a 1×1 kernel with no padding and the ReLU activation function.

The pooling layers L3, L6, L9 are max-pooling layers, replacing four neighboring nodes with only one node, the value being the maximum of the values of the four neighboring nodes. The upsampling layers L12, L15, L18 are transposed convolution layers with 3×3 kernels and stride 2, which effectively quadruple the number of nodes. The dashed horizontal arrows correspond to concatenation operations, where the output of a convolutional layer L2, L5, L8 of the downsampling branch of the U-Net structure is used as additional inputs for a convolutional layer L13, L16, L19 of the upsampling branch of the U-Net structure. This additional input data is treated as additional channels in the input node layer for the convolutional layer L13, L16, L19 of the upsampling branch.

For training the CNN, a database of 500 first medical images was used, wherein the respective segmentation mask was created based on annotations of expert radiologists. In particular, the experts determined for each of the 500 first medical images a segmentation mask for a structure of interest, where a value of 1 was assigned to pixels corresponding to the structure of interest, and a value of 0 was assigned to pixels not corresponding to the structure of interest. The database was split into training data (320 datasets), validation data (80 datasets) and test data (100 datasets). For training the CNN, the backpropagation algorithm was used based on a binary cross-entropy cost function

$$L ( x , y ) = {\sum }_{i} {\sum }_{j} BCE ( y [ i , j ] . M ( x ) [ i , j ] )$$

with

$$BCE ( a , b ) := - a log ( b ) ( b ) - ( 1 - a ) log ( 1 - b ) ,$$

wherein x denotes a first medical image, y determines the corresponding segmentation mask created by the expert radiologist, and M(x) denotes the result of applying the CNN to the first input medical image x. Alternatively, one could use other cost functions like weighted binary cross entropy, Focal Loss or Dice Loss.

Based on the validation set of 80 datasets and the corresponding annotations, the best performing machine learning model out of several machine learning models (with different hyperparameters, for example number of layers, size and number of kernels, padding et cetera) was selected. The specificity and the sensitivity were determined based on the test set comprising 100 datasets and the corresponding annotations.

Independent of the grammatical term usage, individuals with male, female or other gender identities are included within the term “patient”.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RA), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium.

Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for imaging-based characterization of a perfusion defect in a vessel structure for blood supply of at least one organ of a patient, the computer-implemented method comprising:
receiving medical imaging data depicting the at least one organ and the vessel structure, wherein the medical imaging data includes energy resolved CT imaging data;
detecting a blood stream obstructing object in the vessel structure, based on the medical imaging data; and
determining, based on the energy resolved CT imaging data, a perfusion defect score for a target region of the at least one organ, whose blood perfusion is potentially affected by the blood stream obstructing object.

2. The computer-implemented method according to claim 1, further comprising:
generating a segmentation dividing the at least one organ into a plurality of segments, based on the medical imaging data, wherein
the plurality of segments are hierarchically classified according to their blood supply by the vessel structure, and
determining, according to the hierarchical classification, one or more target segments of the plurality of segments as the target region, the one or more target segments having blood perfusion that is potentially affected by the blood stream obstructing object.

3. The computer-implemented method according claim 2, wherein
for each of the one or more target segments, a respective segment perfusion defect score is determined based on the energy resolved CT imaging data, and
the perfusion defect score is determined based on the segment perfusion defect scores.

4. The computer-implemented method according to claim 3, wherein
for each of the one or more target segments, a size of a perfusion defect region in a respective target segment is determined based on the energy resolved CT imaging data, and
the respective segment perfusion defect score is determined based on the size of the perfusion defect region.

5. The computer-implemented method according to claim 2, wherein the segmentation is generated by applying a first trained machine learning model to first input data including the medical imaging data.

6. The computer-implemented method according to claim 1, wherein
at least one perfusion blood volume value for the target region is determined depending on the energy resolved CT imaging data, and
the perfusion defect score is determined based on at least one perfusion blood volume value.

7. The computer-implemented method according to claim 6, wherein the at least one perfusion blood volume value includes a respective segment perfusion blood volume value for one or more target segments.

8. The computer-implemented method according to claim 1, wherein
the medical imaging data includes photon-counting CT imaging data, and
the blood stream obstructing object is detected based on the photon-counting CT imaging data.

9. The computer-implemented method according to claim 1, wherein the blood stream obstructing object is detected by applying a trained machine learning model to input data including the medical imaging data.

10. The computer-implemented method according to claim 1, wherein the energy resolved CT imaging data includes contrast enhanced CT imaging data.

11. The computer-implemented method according to claim 1, wherein the perfusion defect score is determined by applying a trained machine learning model to input data including the medical imaging data.

12. The computer-implemented method according to claim 1, wherein the at least one organ includes lungs of the patient.

13. A data processing system configured to perform the computer-implemented method according to claim 1.

14. A medical imaging system comprising:
the data processing system according to claim 13; and
a CT device configured to generate the medical imaging data depicting the at least one organ and the vessel structure.

15. A non-transitory computer-readable storage medium storing computer-executable instructions that, when executed by a data processing system, cause the data processing system to perform the computer-implemented method of claim 1.

16. The computer-implemented method according to claim 4, wherein the segmentation is generated by applying a first trained machine learning model to first input data including the medical imaging data.

17. The computer-implemented method according to claim 4, wherein
at least one perfusion blood volume value for the target region is determined depending on the energy resolved CT imaging data, and
the perfusion defect score is determined based on at least one perfusion blood volume value.

18. The computer-implemented method according to claim 4, wherein
the medical imaging data includes photon-counting CT imaging data, and
the blood stream obstructing object is detected based on the photon-counting CT imaging data.

19. The computer-implemented method according to claim 4, wherein the blood stream obstructing object is detected by applying a trained machine learning model to input data including the medical imaging data.

20. The computer-implemented method according to claim 4, wherein the perfusion defect score is determined by applying a trained machine learning model to input data including the medical imaging data.
